(19) United States
(12) Patent Application Publication
(10) Pub. No.: US 2009/0011058 A1
(43) Pub. Date: Jan. 8, 2009

(54) Standardized extract of the peppercorns from the plant zanthoxylum pipertium(Hua Jiao or Sansho) used to manage, treat and/or reverse autism, autism, spectrum disorders, autistic-like symptoms, Fragile-X, and/or Fragile-X symptoms (FXS) by seleltively blocking and halting the enzymatic activity of the kinase PAK

(76) Inventors: Patricia Ann Carlson, San Antonio, TX (US); Scott Carlson, San Antonio, TX (US)

Correspondence Address: Scott Carlson, 3242 Monarch, San Antonio, TX, 78259, US

(21) Appl. No.: 12/069,465

(22) Filed: Feb. 11, 2008

Related U.S. Application Data
(60) Provisional application No. 60/958,028, filed on Jul. 2, 2007.

(51) Int. Cl. A61K 36/67; A61P 25/00

(52) U.S. Cl. 424/734

(57) ABSTRACT

This invention provides a standardized extract of the peppercorns from the plant Zanthoxylum pipertium (Hua Jiao or Sansho). The extract provides an all natural, side effect free method to manage, treat and/or reverse autism, autism spectrum disorders, autistic-like symptoms, Fragile-X, and/or Fragile-X symptoms (FXS) by selectively blocking and halting the enzymatic activity of the kinase PAK.

### CROSS-REFERENCE TO RELATED APPLICATIONS

This nonprovisional utility patent application is a continuation of, and seeks the benefit of, the provisional patent application No. 60/958,028, filed on Jul. 02, 2007, by Patricia Carlson and Scott Carlson, pending, which is incorporated by reference as if fully set forth herein.

A standardized extract of the peppercorns from the plant Zanthoxylum pipertium (Hua Jiao and Sansho) used to manage, treat and/or reverse autism, autism spectrum disorders, autistic-like symptoms, Fragile-X, and/or Fragile-X symptoms (FXS) by selectively blocking and halting the enzymatic activity of the kinase PAK, thereby reducing the symptoms and/or reversing autism, autistic spectrum disorders, and Fragile-X symptoms (FXS), including, but not limited to hyperactivity, repetitive movements, attention deficits, and memory challenges. The standardized dosage shall vary by a person's age and weight.

Dosage for either extract will be 110 mg/kg. If the dose consists of Hua Jiao the concentration of the extract will be 0.02 mg/ml. If the dose consists of Sansho peppercorn extract, the concentration will be 0.1 mg/ml.

### STATEMENT REGARDING FEDERALLY SPONSORED RESEARCH

Not Applicable

### REFERENCE TO SEQUENCE LISTING, TABLE, OR A COMPUTER PROGRAM LISTING

Not Applicable

### BACKGROUND OF THE INVENTION

The present invention pertains to non-obvious and novel nutritional supplements. The supplements are comprised of extracts from either the Chinese peppercorn known as “Hua Jiao” or the Japanese peppercorn known as “Sansho.” The extracts in concentrations and dosages referenced herein are capable of managing, treating and/or reverse autism, autism spectrum disorders, autistic-like symptoms, Fragile-X, and/or Fragile-X symptoms (FXS) by selectively blocking and halting the enzymatic activity of the kinase PAK. As such, this invention also pertains to an all natural, side effect free method of managing, treating and/or reverse autism, autism spectrum disorders, autistic-like symptoms, Fragile-X, and/or Fragile-X symptoms (FXS).

The fragile X syndrome is a genetic disorder caused by mutation of the FMR1 gene on the X chromosome. The National Institute of Health reports that the mutation at that site is found in 1 out of about every 4000 males and 1 out of about every 8000 females. See http://www.med.umich.edu/1libr/yourchild/fragilez.htm. Normally, the FMR1 gene contains between 6 and 55 repeats of the CGG codon (trinucleotide repeats). In people with the fragile X syndrome, the FMR1 allele has over 230 repeats of this codon. Expansion of the CGG repeating condon to such a degree results in a methylation of that portion of the DNA, effectively silencing the expression of the FMR1 protein. This methylation of the FMR1 locus in chromosome band Xq27.3 is believed to result in constriction of the X chromosome which appears ‘fragile’ under the microscope at that point, a phenomenon that gave the syndrome its name.

Mutation of the FMR1 gene leads to the transcriptional silencing of the fragile X-mental retardation protein, FMRP. In normal individuals, FMRP attaches and promotes the translation of many essential neuronal RNAs. In fragile X patients, however, these RNAs are not translated into proteins. Fragile X syndrome is the most common form of inherited mental retardation and autism. See http://www.med.umich.edu/1libr/yourchild/fragilex.htm. People with only a small change in the gene might not show any signs of Fragile X. People with bigger changes can have severe symptoms. These might include intelligence problems, sensory issues, such as anxiety, attention deficits, aggression, shyness, and speech and language problems. Fragile X syndrome can cause a child to have autism or an Autism Spectrum Disorder (ASD), though not all children with fragile X syndrome have autism or an ASD.

The term “Autism” as used herein, includes classic autism, autism spectrum disorders, pervasive developmental disorders (PDD), and pervasive developmental disorders not otherwise specified (PDDNOS). Autism is a disorder that is usually first diagnosed in early childhood. As described in DSM IV, Section 2999.00, the main signs and symptoms of autism involve communication, social interactions, and repetitive and/or obsessive behaviors. The symptoms of autism can usually be observed by 18 months of age. As indicated by the National Institute of Health, children with autism might have problems talking with you, or they might not look you in the eye when you talk to them. They may line objects up before the can pay attention, or they may same the sentence again and again to calm themselves down. They may flap their arms to tell you they are happy, or they might hurt themselves to indicate to you that they are not. Some people with autism never speak and never become toilet trained.

Many physicians are currently treating symptoms associated with fragile-x, Fragile-X Syndrome, autism, and the symptoms associated with Autism Spectrum Disorder with pharmaceuticals. For instance, anxiety and depression are being treated with selective serotonin reuptake inhibitors such as Prozac. Behavioral problems are often treated with antipsychotic medications such as Risperdone, Zyprexa, Geodon, Haldol, fluphenazine, and chlorpromazine. Seizures are treated with Tegretol, Lamictal, Topamax, or Depakote. Inattention and hyperactivity are treated with Ritalin, Concentra and Adderall. All of the aforementioned medications, however, have the potential for side effects. Some of which are serious and sometimes permanent. Moreover, none of the aforementioned pharmaceuticals address the cause of fragile X, fragile X syndrome, autism, and/or Autism spectrum Disorders. Rather, they are only intended to manage symptoms. Consequently, a safer, more natural alternative to address the cause of these conditions would be desirable.

Recently, research has indicated that autism, Autism Spectrum Disorders, and Fragile X Syndrome, and the symptoms associated therewith, may be corrected, and reversed by inhibiting the activity of p21-activated kinase (PAK). See Hayashi, Rao, Seo, Choi, Dolan, Choi, Chattarji, and Tonegawa; Inhibition of p21-Activated Kinase Rescues Symptoms of Fragile X-Syndrome in Mice, www.pnas.org/cgi/doi/10.1073/pnas.0705003104. There are drugs on the market which have some PAK inhibiting properties, such as the anti-epileptic drug valproate, but these drugs have side effects which are not desirable. This invention proposes using potent, all natural PAK inhibitors which have no side effects. Furthermore, the invention is intended to address the underlying causes of autism, rather than merely treating or masking the symptoms.

Specifically, the extract from the Chinese peppercorn Hua Jiao, and the extract from the Japanese peppercorn Sansho have been shown to be effective in treating cancer by down regulating cyclin D1. See Hirokawa, Nheu, Grimm, Mautner, Maeda, Yoshida, Komiyama, and Maruta, Schuan Pepper Extracts Block the PAK1/Cyclin D1 Pathway and the Growth of NF1-Deficient Cancer Xenograft in Mice, Cancer Biology & Therapy 5:3, 305-309, March 2006. This down regulation was accomplished because the extract of Hua Jiao and Sansho peppercorns has been shown to selectively block the kinase PAK. Id. This invention proposes using a standardized extract of the very same peppercorns to treat autism, Autism Spectrum Disorders, fragile x, and fragile x syndrome. This may be accomplished by using the extract to inhibit the activity of p21-activated kinase (PAK). This in turn will eliminate behavioral deficits, eliminate anxiety, calm hyperactivity, reduce or eliminate repetitive behaviors, improve memory, restore locomotion, and facilitate socialization.

### SUMMARY OF THE INVENTION

A standardized extract of the peppercorns from the plant Zanthoxylum pipertium (Hua Jiao and Sansho) used to manage, treat and/or reverse autism, autism spectrum disorders, autistic-like symptoms, Fragile-X, and/or Fragile-X symptoms (FXS) by selectively blocking and halting the enzymatic activity of the kinase PAK, thereby reducing the symptoms and/or reversing autism, autistic spectrum disorders, and Fragile-X symptoms (FXS), including, but not limited to hyperactivity, repetitive movements, attention deficits, and memory challenges.

### BRIEF DESCRIPTION OF THE SEVERAL VIEWS OF THE DRAWING

Not applicable.

### DETAILED DESCRIPTION OF THE INVENTION

A standardized extract of the peppercorns from the plant Zanthoxylum pipertium (Hua Jiao and Sansho) used to manage, treat and/or reverse autism, autism spectrum disorders, autistic-like symptoms, Fragile-X, and/or Fragile-X symptoms (FXS) by selectively blocking and halting the enzymatic activity of the kinase PAK, thereby reducing the symptoms and/or reversing autism, autistic spectrum disorders, and Fragile-X symptoms (FXS), including, but not limited to hyperactivity, repetitive movements, attention deficits, and memory challenges.

The extract may be obtained in the manner set forth below or in a similar fashion.

1. Hua Jiao
a. Soak 100 g of Hua Jiao in 400 ml of 70% ethanol in water (v/v)
b. Extract the anti-PAK activity by rotating a 500 ml container for 3-7 hours at a temperature of 25 degrees Celsius;
c. Filter the peppercorns and centrifuge the filtrate at 200 rpm for 5 minutes to remove the leftover insoluble powder.

2. Sansho
a. Take 5 g of ground peppercorns and suspend in 50 ml of 1% HCL in methanol (v/v)
b. Extract the anti-PAK ingredients by soaking the mixture in a 45 degree Celsius water bath for thirty second every ten minutes for one hour
c. Centrifuge the mixture at 2000 rpm for 5 minutes
d. The extract then must be freeze-dried in a vacuum (lyophilized) to remove the HCL and methanol, and then re-extracted with 10 ml of 70% ethanol in water (v/v) at room temperature.
   Both extracts should be kept either refrigerated or frozen until use. The extracts can remain in liquid form, or they can be encapsulated. In either event, the extract is taken orally.

The standardized dosage shall vary by a person's age and weight. Dosage for either extract will be 110 mg/kg. If the dose consists of Hua Jiao the concentration of the extract will be 0.02 mg/ml. If the dose consists of Sansho peppercorn extract, the concentration will be 0.1 mg/ml. Obviously, it will be possible to combine the extracts of the Hua Jiao and Sansho in the above referenced concentrations to achieve the recommended dosage. This dosage shall serve as a guide. It is possible that the effective dosage may vary from individual based upon such factors as age, weight and severity of symptoms.

There are drugs on the market which have some PAK inhibiting properties, such as the anti-epileptic drug valproate, but these drugs all have unpleasant side effects. This invention is an improvement on the current state of the art because it proposes using potent, all natural PAK inhibitors which have no adverse side effects. This invention is distinguished from prior invention, and is an improvement on the current state of the art because it provides a natural, side effect free method to manage, treat and/or reverse autism, autism spectrum disorders, autistic-like symptoms, Fragile-X, and/or Fragile-X symptoms (FXS) by reducing the symptoms and/or reversing autism, autistic spectrum disorders, and Fragile-X symptoms (FXS), including, but not limited to hyperactivity, repetitive movements, attention deficits, and memory challenges. Furthermore, unlike pharmaceuticals which comprise the current state of the art, this invention treats the underlying cause of the disorders and does not merely mask symptoms.

The extract made the subject of this patent will achieve their intended purposes by inhibiting the catalytic activity of PAK (p21-activated kinase). This kinase is known to play a role in fragile-x, fragile-x syndrome, autism and Autism Spectrum Disorders. See Hayashi, Rao, Seo, Choi, Dolan, Choi, Chattarji, and Tonegawa; Inhibition of p21-Activated Kinase Rescues Symptoms of Fragile X-Syndrome in Mice, www.pnas.org/cgi/doi/10.1073/pnas.0705003104. By blocking the PAK1 signal pathway, the invention is able to manage, treat and/or reverse autism, autism spectrum disorders, autistic-like symptoms, Fragile-X, and/or Fragile-X symptoms (FXS) by selectively blocking and halting the enzymatic activity of the kinase PAK, thereby reducing the symptoms and/or reversing autism, autistic spectrum disorders, and Fragile-X symptoms (FXS), including, but not limited to hyperactivity, repetitive movements, attention deficits, and memory challenges.

1. We claim a method of managing autism, autism spectrum disorders, autistic-like symptoms by selectively blocking and halting the enzymatic activity of the kinase PAK, thereby reducing the symptoms and/or reversing autism, and autistic spectrum disorders including, but not limited to hyperactivity, repetitive movements, attention deficits, and memory challenges.
We claim a method of managing Fragile-X, and/or Fragile-X symptoms (FXS) by selectively blocking and halting the enzymatic activity of the kinase PAK, thereby reducing the symptoms and/or reversing autism, and autistic spectrum disorders including, but not limited to hyperactivity, repetitive movements, attention deficits, and memory challenges.
We further claim method of treating autism, autism spectrum disorders, autistic-like symptoms by selectively blocking and halting the enzymatic activity of the kinase PAK, thereby reducing the symptoms and/or reversing autism, and autistic spectrum disorders including, but not limited to hyperactivity, repetitive movements, attention deficits, and memory challenges.
We claim a method of treating Fragile-X, and/or Fragile-X symptoms (FXS) by selectively blocking and halting the enzymatic activity of the kinase PAK, thereby reducing the symptoms and/or reversing autism, and autistic spectrum disorders including, but not limited to hyperactivity, repetitive movements, attention deficits, and memory challenges.
We also claim a method of reversing autism, autism spectrum disorders, autistic-like symptoms by selectively blocking and halting the enzymatic activity of the kinase PAK, thereby reducing the symptoms and/or reversing autism, and autistic spectrum disorders including, but not limited to hyperactivity, repetitive movements, attention deficits, and memory challenges.
Additionally, we claim a method of reversing Fragile-X, and/or Fragile-X symptoms (FXS) by selectively blocking and halting the enzymatic activity of the kinase PAK, thereby reducing the symptoms and/or reversing autism, and autistic spectrum disorders including, but not limited to hyperactivity, repetitive movements, attention deficits, and memory challenges.
We claim a standardized extract of the peppercorns from the plant Zanthoxylum pipertium (Hua Jiao) used to manage, treat and/or reverse autism, autism spectrum disorders, autistic-like symptoms, Fragile-X, and/or Fragile-X symptoms (FXS) by selectively blocking and halting the enzymatic activity of the kinase PAK, thereby reducing the symptoms and/or reversing autism, autistic spectrum disorders, and Fragile-X symptoms (FXS), including, but not limited to hyperactivity, repetitive movements, attention deficits, and memory challenges.
We claim a standardized extract of the peppercorns from the plant Zanthoxylum pipertium (Sansho) used to manage, treat and/or reverse autism, autism spectrum disorders, autistic-like symptoms, Fragile-X, and/or Fragile-X symptoms (FXS) by selectively blocking and halting the enzymatic activity of the kinase PAK, thereby reducing the symptoms and/or reversing autism, autistic spectrum disorders, and Fragile-X symptoms (FXS), including, but not limited to hyperactivity, repetitive movements, attention deficits, and memory challenges.
